(19) United States
(12) Patent Application Publication
(10) Pub. No.: US 2026/0351392 A1
(43) Pub. Date: Nov. 5, 2026

(54) Hybrid Asset Tracking Network for Surgical Tools

(71) Applicant: Peritas AI Inc., Fort Lauderdale, FL (US)

(72) Inventors: Martin Roche, Fort Lauderdale, FL (US); Joseph DeCerce, Fort Lauderdale, FL (US)

(21) Appl. No.: 19/637,235

(22) Filed: Apr. 2, 2026

Related U.S. Application Data
(60) Provisional application No. 63/782,696, filed on Apr. 3, 2025.

(51) Int. Cl. G16H 40/20

(52) U.S. Cl. CPC G16H 40/20

(57) ABSTRACT

The present disclosure relates to a hybrid asset tracking network for the efficient management and tracking of surgical tools, sterilization trays and carts within a hospital environment. The network incorporates rugged local trackers affixed to each sterilization tray capable of withstanding autoclave sterilization temperatures. These local trackers are paired with global trackers on carts for broad-area localization of tools throughout the hospital. The system also includes local and global receivers, an integrated network that registers the presence and status of tools at checkpoints, and an optical system with computer vision capabilities for verifying tool contents during assembly. This hybrid network improves and enhances the workflow associated with sterilization and utilization of surgical tools, ensuring tools are readily available, sterilized, and accurately tracked across the hospital.

### TECHNICAL FIELD

The disclosure pertains to an asset tracking system in a hospital setting that tracks the location of surgical tools, sterilization trays, and carts.

### BACKGROUND

The management of surgical tools in a hospital environment poses many logistical and safety challenges. These tools must be meticulously cleaned, sterilized, and tracked to prevent infections and ensure that surgical procedures are not delayed due to missing or unprepared equipment. Traditional systems for managing surgical tools often rely on manual tracking methods, which can be error-prone and inefficient. These methods may also fail to provide real-time tracking and status updates for surgical instruments, leading to increased risk of procedural delays and potential harm to patients.

Existing tracking technologies, like Radio-Frequency Identification (RFID) and barcode scanning, have been employed in hospitals to track assets. However, these technologies have significant limitations. For instance, many RFID systems are not able to withstand the extreme temperatures associated with autoclave sterilization, and barcodes require line-of-sight and may be damaged or become unreadable after repeated sterilization cycles. Furthermore, typical RFID systems that operate at high frequencies are unable to transmit efficiently through metallic surfaces, making them ill-suited for tracking metal surgical tools housed within metallic sterilization trays.

In light of these challenges, there remains a need for an improved tracking system capable of operating reliably in the harsh conditions experienced during the sterilization of surgical tools and providing accurate location data for tools and trays as they are moved throughout various parts of the hospital.

### SUMMARY

The present disclosure relates generally to systems and methods to improve the tracking, management, and verification of surgical tools within a hospital. Specifically, this disclosure pertains to developing a hybrid asset tracking network capable of withstanding the rigorous environment of sterilization processes and providing precise location information within a hospital setting.

The present disclosure relates generally to asset management systems, and more particularly to a hybrid asset tracking network designed for the management and tracking of surgical tools and trays within a hospital environment. This system ensures the traceability, sterilization status confirmation, and location monitoring of surgical assets to enhance operational efficiency and maintain stringent standards of sterilization.

The disclosed hybrid asset tracking network integrates both local and global tracking technologies. The system encompasses a series of rugged local trackers affixed to sterilization trays, which are capable of enduring the severe conditions of autoclave sterilization, including temperatures of at least 250° F. These trackers are equipped with necessary sensors to confirm the integrity of sterilization cycles and to detect moisture indicative of wet load conditions-all without disturbing the integrity of sterile wraps.

Complementing the local trackers are global trackers located on carts that transport the sterilization trays. These trackers facilitate the broad-area localization of surgical tools as they are moved across various departments and operating rooms within the hospital. Utilization of distinct frequency bands aids in the differentiation and optimization of the two tracking systems, with local trackers functioning on low frequency bands and global trackers operating on ultra high or super high frequency bands.

Furthermore, the system incorporates local and global receivers, strategically placed to ensure thorough tracking coverage in areas of critical relevance, such as sterilization facilities and operating rooms. A network aggregates the received signals and registers the presence and status of the sterilization trays when they pass specific checkpoints containing both kinds of receivers.

One aspect of the disclosure includes an assembly table embedded with a local receiver, which cooperates with an optical system endowed with computer vision capabilities. This system is programmed to identify individual surgical tools, which allows for real-time verification of tray contents during the assembly process. An associated database maintains updated records of the tools based on identifications made by the optical system.

The network system includes a processing means to evaluate the signal strengths transmitted from the trackers, thereby facilitating the update of the database infrastructure with accurate and current location information. This aids in comprehensive hospital-wide tracking that encompasses dynamic records of tray and cart positions and reconciles these with the sterilization status to ensure readiness for operative procedures.

The present disclosure also contemplates several methodological aspects reflecting the application of local and global tracking in the hospital environment. It encompasses methods for tracking sterilization trays, managing location and sterilization data, verifying the presence and sterile status of the trays, and identifying wet load conditions.

1n summary, the proposed hybrid asset tracking network significantly streamlines the workflow associated with the sterilization and utilization of surgical tools, thereby increasing efficiency and reliability, diminishing the risk of infection, and optimizing the availability of tools when and where they are needed most within the hospital setting.

### BRIEF DESCRIPTION OF DRAWINGS

For a better understanding of the disclosure, its operating advantages, and the specific objects attained by its uses, reference should be made to the accompanying drawings and descriptive matter in which there are illustrated example embodiments of the disclosure.

FIG. 1 is a schematic diagram illustrating an overview of the hybrid asset tracking network system deployed within a hospital environment.

FIG. 2 is a detailed perspective view of a sterilization tray equipped with a local tracker capable of withstanding high temperatures and a local receiver positioned near a sterilization area.

FIG. 3 is a perspective view of a cart equipped with a global tracker for transporting sterilization trays between different hospital departments, including operating rooms.

FIG. 4 is a block diagram representing the signal communication between local trackers on sterilization trays and local receivers near sterilization areas.

FIG. 5 is a block diagram depicting the communication between global trackers on carts and global receivers within the hospital operating rooms.

FIG. 6 is a flowchart describing the steps involved in the asset tracking method within a hospital environment, including the use of local and global trackers and a network to collect data.

FIG. 7 is a top view of an assembly table configured with an integrated local receiver for identifying sterilization trays placed upon it.

FIG. 8 is a schematic representation of an optical system equipped with computer vision capabilities, used to identify surgical tools on the assembly table.

FIG. 9 is a generalized schematic diagram of the database network connected to the optical system for updating inventory records corresponding to identified surgical tools.

FIG. 10 is a schematic representation of the interconnected network system analyzing signal strength and updating the database with location information of sterilization trays and carts.

In the drawings and specification, there have been disclosed typical preferred embodiments of the disclosure, and although specific terms are employed, they are used in a generic and descriptive sense only and not for purposes of limitation.

### DETAILED DESCRIPTION

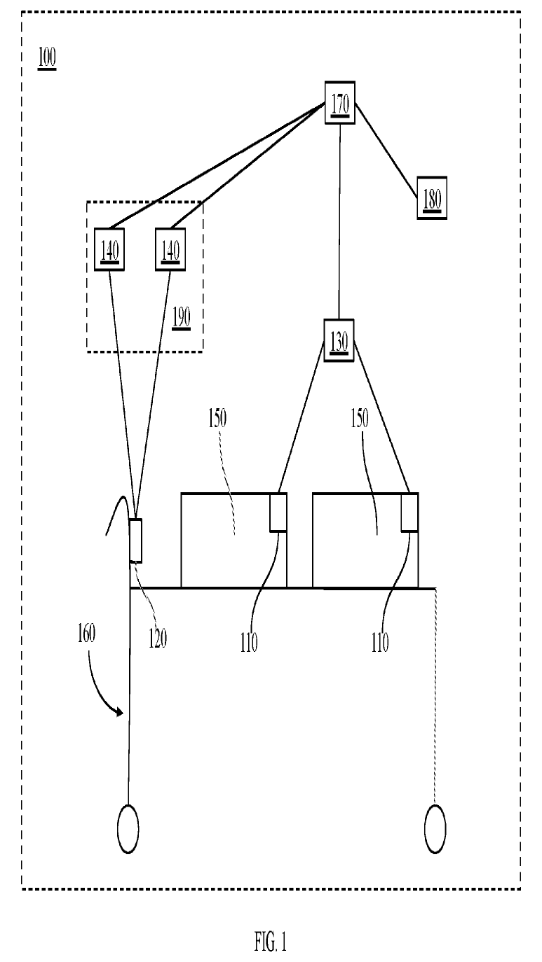

Referring now more specifically to the drawings, FIG. 1 illustrates a hybrid asset tracking network system 100, deployed within a hospital environment to manage and track surgical tools and trays. The hybrid asset tracking network system 100 includes a plurality of components namely, local trackers 110, global trackers 120, local receivers 130, global receivers 140, sterilization trays 150, carts 160, an interconnecting network 170, an optical system 180 and a localization system 190.

Local trackers 110 are ruggedized devices affixed to sterilization trays 150, designed to withstand the extreme temperatures encountered during the autoclave sterilization process, specifically at least 250° F. These trackers are equipped with sensors to validate the completion of the sterilization cycle and to detect moisture levels, each contributing to ensuring that surgical tools meet the required standards for safety and sterilization.

Global trackers 120, are affixed to carts 160, which are used for transporting sterilization trays 150 between various hospital departments, such as surgical wards and operating rooms. These trackers are characterized by their ability to transmit data across broad distances, at least 40 meters, operating primarily on ultra high frequency or super high frequency bands to facilitate wide-area localization of surgical tools and trays within the hospital. As used herein, low frequency, ultra high frequency and super high frequency refer to respective ITU designations.

Local receivers 130 are positioned in close proximity to areas subjected to high temperatures and moisture, wherein sterilization trays pass through during the sterilization processes. They communicate with local trackers 110 to register the presence and conditions of the trays.

Global receivers 140 are installed in several strategic locations throughout the hospital, especially within or near operating rooms, enabling them to detect signals from global trackers 120 on the carts 160. This allows for the effective monitoring of the location of those carts within the hospital. In one embodiment global trackers 120 and global receivers 140 are Bluetooth Low Energy trackers and receivers, respectively, conforming to IEEE standard 802.15. In another embodiment global trackers 120 and global receivers 140 are Wi-Fi trackers and receivers, respectively, conforming to IEEE standard 802.11.

The interconnecting network 170 serves as the central platform that receives, aggregates, and processes signal information from both types of receivers, local 130 and global 140. As such, it plays an important role in registering the presence of sterilization trays 150 when they reach certain checkpoints endowed with both local and global receivers.

The network 170 includes processing means to examine the strength and nature of the signals received from local trackers 110 and global trackers 120. This data processing informs the effective updating of a database infrastructure (not shown in FIG. 1) with real-time information on the location and sterilization status of the trays and carts, ensuring that surgical instruments are ready and available for procedures within the hospital.

A feature of the hybrid asset tracking network system 100 represented in FIG. 1 is its duality in operation, where local tracking is well-suited for close-range, highly specific applications, such as verification of appropriate sterilization parameters and environmental conditions, while global tracking operates effectively over a longer range to generally locate and manage the assets within the hospital environment. Together, these systems coalesce to form a comprehensive asset management solution, enhancing the operational efficiency, safety, and preparedness in hospital settings.

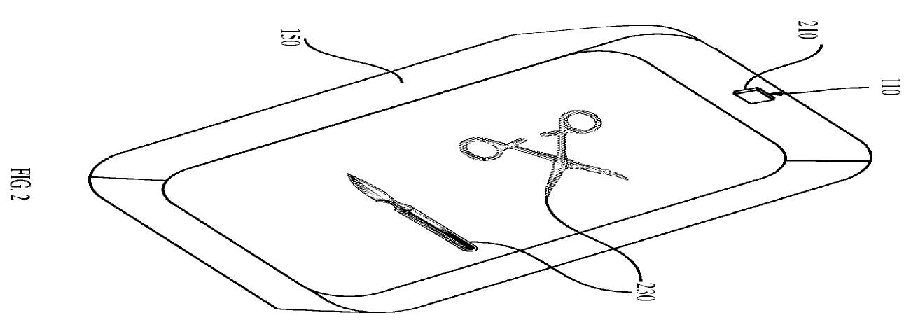

Turning now to FIG. 2 of the drawings, there is shown a detailed perspective view of a sterilization tray 150, which is equipped with a local tracker 110. The local tracker 110 includes a durable housing 210 that is constructed to endure the harsh conditions of sterilization processes, including exposure to high temperatures that typically exceed 250° F. In addition, the local tracker 110 encompasses a battery unit (not shown) optimized for extended life and performance stability in extreme thermal environments. In one embodiment, the battery life of the local tracker 110 is at least five years.

A local receiver 130 is positioned in approximate adjacency to the sterilization area to enable the detection of local trackers 110 when trays 150 are present. The local receiver 130, being part of the close-range component of the hybrid asset tracking network, communicates with the local tracker 110 utilizing low frequency signals that penetrate the harsh surroundings of the sterilization environment. The low frequency transmission is particularly advantageous as it can effectively traverse metallic and water-rich barriers, contributing to reliable communication even when sterilization trays are enveloped by metal, dense steam and hot air.

The sterilization tray 150 is depicted with an arrangement of surgical tools 230, signifying an exemplary payload potentially contained during processing. The tray 150 is typically fabricated from a metal compatible with both the thermal aspects of sterilization.

Incorporated into the design of the local tracker 110 is a temperature sensor that plays a pivotal role in confirming that a sterilization cycle has reached the required sterilizing temperature. By continuously monitoring the temperature, the local tracker 110 contributes to the validation of the sterilization process, ensuring that each tool 230 on the tray 150 has been subjected to appropriate sterilization conditions, thereby maintaining hospital hygiene and patient safety standards.

The local tracker 110, in some embodiments, can also be configured to include a humidity sensor. This sensor aims to detect moisture levels indicative of wet load conditions, which can compromise the sterility of surgical tools 230. By incorporating this feature, the local tracker 110 is further empowered to alert hospital staff of any anomalies in the expected sterilization outcomes.

The design elements of the local tracker 110 are chosen to withstand repeated cycles of autoclave sterilization without affecting the functionality or structural integrity of the device. Such design considerations extend to the selection of robust materials for the housing 210, battery, and electronic components (e.g., temperature and humidity sensors), which together contribute to the longevity and reliability of the tracking system.

The close proximity of local receiver 130 to the sterilization area helps to ensure stable communication with the local tracker 110 as soon as the sterilization tray 150 is removed from the autoclave or similar sterilization device. The local receiver 130, being network-enabled, promptly relays status information including but not limited to, temperature readings, humidity levels, and tray presence to the interconnected network 170. This real-time data transfer enables the network to keep an up-to-date record of the sterilization status and to readily disseminate this information to relevant hospital personnel and systems.

This embodiment of the hybrid asset tracking network not only recognizes the presence of the trays within sterilization areas but also closely monitors the critical parameters that directly affect the efficacy of the sterilization process, helping to ensure the sustained integrity and readiness of the surgical tools 230 contained within each sterilization tray 150.

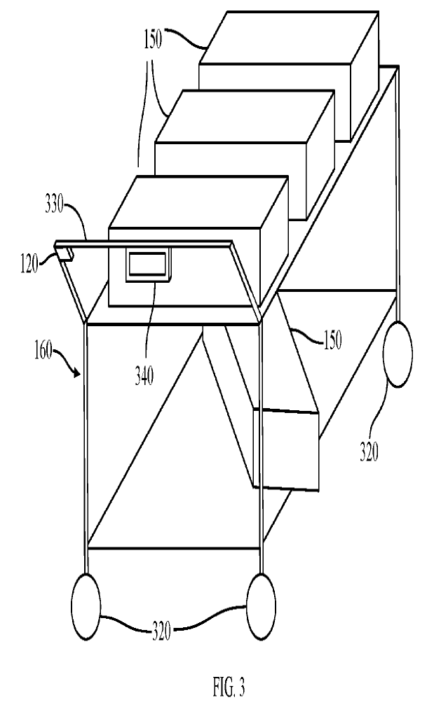

FIG. 3 illustrates a perspective view of a cart 160, which is a component of the hybrid asset tracking network system 100. The cart 160 is specifically designed to securely transport sterilization trays 150 between various departments within the hospital, such as from sterilization facilities to operating rooms, or to storage areas for ready-to-use surgical equipment.

The cart 160 is shown equipped with a global tracker 120, which is a device responsible for the broader area tracking within the asset tracking network system 100. The global tracker 120 includes a communication module housed within a protective casing 310 that provides resistance to mechanical stress and environmental factors commonly encountered during normal hospital operations. The global tracker 120 is capable of transmitting data over considerable distances, at least 40 meters, to provide consistent connectivity with a network of global receivers 140 placed in strategic locations throughout the hospital.

A unique identifier associated with the global tracker 120 allows for the distinct identification of each cart 160 as it traverses through various points within the hospital infrastructure. This identifier facilitates location tracking, enabling accurate and up-to-date information on the whereabouts of each cart and the associated sterilization trays 150.

The global tracker 120 operates on a frequency band that is distinct from that used by the local trackers 110, which may include ultra high frequency (UHF) or super high frequency (SHF) bands. The choice of frequency band for the global tracker 120 is determined based on factors such as the need for signal penetration through building materials, signal range, data transmission rate requirements, and potential interference from other hospital equipment.

In the depicted embodiment, the cart 160 includes features such as wheels 320 for maneuverability and a handle 330 for ergonomic transport. The wheels 320 are designed to easily navigate hospital corridors and elevators, ensuring smooth and efficient delivery of sterilization trays to their intended destinations.

The construction of the cart 160 is mindful of the ease of cleaning and its ability to support the weight of loaded sterilization trays. Materials for the cart 160 are chosen based on durability, ease of sanitation, and compatibility with hospital environments.

In one embodiment, mounted on the cart 160, in a clearly visible location, is a display unit 340 (e.g., an LED display or LCD screen). The display unit 340 may present important information such as the current status of the cart's load, the presence of sterilization trays 150 on the cart, scheduled delivery destinations, or alerts/notifications pertinent to the surgical assets being transported.

The cart 160 is also capable of interacting with both the global and local receivers, ensuring continuous tracking throughout its entire route. When the cart 160 passes a checkpoint equipped with both global and local receivers, the integrated network 170 registers the carts' presence and, from the received signals of the local trackers 110 on the trays 150, confirms the sterilization status. Such redundancy ensures that the movements and conditions of both equipment types (trays and carts) are meticulously monitored, synergistically contributing to the operational efficacy and safety procedures within the hospital setting.

The global tracker 120 is designed to function in tandem with the aforementioned local tracker 110, thereby sustaining a comprehensive perspective of the assets' locations and statuses at each stage of their transit through the hospital infrastructure. This design underscores the asset tracking system's adaptability and scalability within varying hospital environments, ensuring that the hybrid asset tracking network system 100 optimally serves the hospital's needs for asset management and sterilization verification.

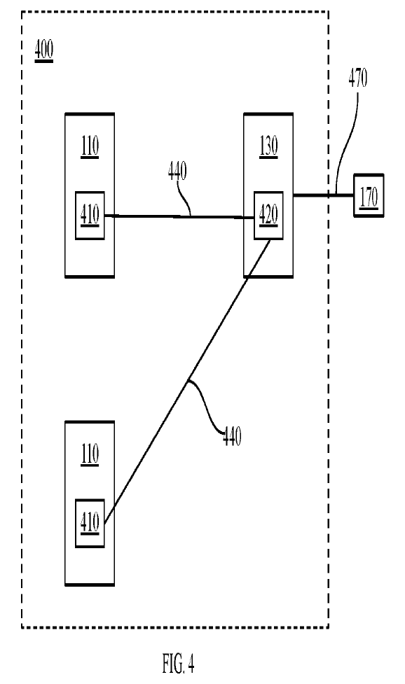

FIG. 4 is a block diagram illustrating the local communication exchange 400 between local trackers 110 on sterilization trays 150 and local receivers 130 located in proximity to sterilization areas within the hospital. In this diagram, multiple sterilization trays, each with an attached local tracker 110, are demonstrated within the sterilization area's field of influence, each executing a specialized communication protocol to interface with corresponding local receivers 130. In one embodiment this specialized communication protocol is RuBee, conforming to IEEE standard 1902.1.

Each local tracker 110 contains a transceiver 410 designed to operate on a low frequency band, emitting signals conducive to penetrating through the challenging environments of a sterilization area, including the presence of metal surfaces, high temperatures, and humidity.

The transceiver 410 is configured to withstand the rigorous conditions experienced during sterilization while maintaining the integrity of data transmission. The local receiver 130 includes a robust receiver module 420 intended to decode signals from local trackers 110 irrespective of environmental interference. The receiver module 420 includes signal capture and processing capability that to isolate and identify signals emitted by each local tracker 110, facilitating an effective inventory and status accounting of each sterilization tray 150 within its range.

The exchange 400 includes an anti-collision protocol, which ensures that local receivers 130 can manage signal traffic from multiple local trackers 110 simultaneously. This protocol aids in resolving any contention for signal bandwidth when numerous sterilization trays 150, each with its local tracker 110, are present within the range of a single local receiver 130. The anti-collision protocol employs algorithms that facilitate clear and uninterrupted communications, even during peak operational periods when many trays 150 are simultaneously in range of local receiver 130.

FIG. 4 also portrays a communication link 440, which represents the flow of data and control instructions between local trackers 110 and local receivers 130. This link 440 illustrates the bi-directional nature of communications, where not only do the local trackers 110 transmit data to local receivers 130, but also they can receive control and configuration updates from receivers 130 as required. In an alternate embodiment, this link is uni-directional with local tracker 110 transmitting data to local receiver 130 receiving data.

The embedded temperature sensor within the local tracker 110 is shown in the diagram as directly influencing the nature of transmitted signals. Specifically, it can alter the signal properties to embed temperature data, which will enable the receiving module 420 to decipher and analyze the thermal history of the tray 150. This data facilitates confirmation that sterilization temperature criteria have been met.

As illustrated in FIG. 4, the local receivers 130 are also connected to the interconnecting network 170 via network connections 470. This linkage enables the real-time data collected by the local receivers 130 to be promptly transmitted to the network system 100 where it is logged, processed, and made accessible to relevant hospital systems and personnel.

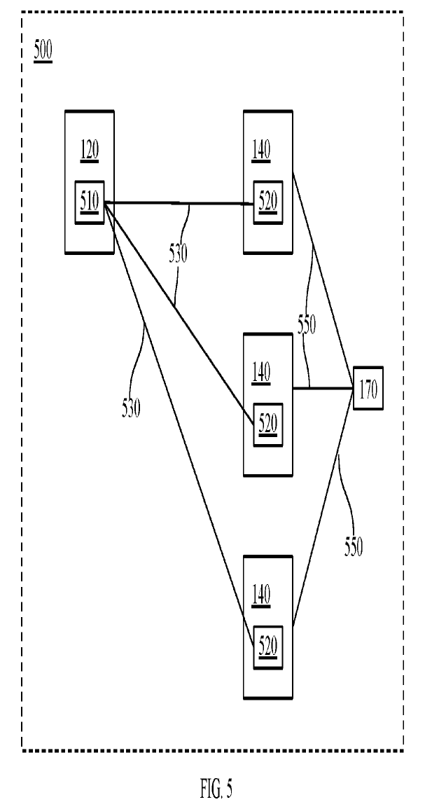

Referring now to FIG. 5, the block diagram illustrates the global communication exchange 500 between the global trackers 120 on the carts 160 and the global receivers 140 located within the operating rooms, halls and other areas of the hospital premises. The global communication exchange 500 in FIG. 5 is primarily concerned with the broad-range aspect of the hybrid asset tracking network system 100, facilitating real-time tracking of the carts 160 as they transport sterilization trays 150 throughout the hospital environment.

Global trackers 120 are affixed to each cart 160 and are designed with a transceiver module 510 which operates on either ultra high frequency (UHF) or super high frequency (SHF) bands. The selection of these higher frequency bands facilitates the communication to cover expansive areas of the hospital, thereby allowing for the continuous and reliable tracking of carts 160 within a more considerable range.

The global receiver 140 incorporates a receiver module 520 that is tuned to receive the signals emitted by the global trackers 120. These receiver modules 520 are strategically distributed in numerous locations across the hospital. In one embodiment the distribution of receiver modules is more concentrated inside or proximate to operating suites. Such placement helps to ensure that the distribution and movement of surgical tools and equipment can be monitored precisely in areas where maintaining a stringent protocol of cleanliness and readiness is paramount.

Each global tracker 120 includes a unique identifier, which is embedded within the transmitted signal 530 that is sent to the global receiver 140. The unique identifier enables the differentiation and identification of individual carts 160, which is crucial for maintaining an accurate and updated log of the movement and location of assets throughout the hospital's various departments.

The global communication system 500 employs a scalable signal processing and anti-collision system, which is adept at handling multiple global trackers 120 operating in the proximity of a global receiver 140. This system ensures that signal overlap or interference between multiple carts 160 does not hinder the effectiveness of the tracking system. The scalability of the system is particularly beneficial as it allows for the incorporation of additional carts and receivers into the network without a significant overhaul of the existing infrastructure.

Global receivers 140 connect with the centralized interconnecting network 170 through network connections 550. The use of these connections 550 indicates how the communication between the global trackers 120 and the global receivers 140 is not an isolated exchange but is integrated into the wider information system of the hospital. Data on the location and movement of carts 160 and their trays 150 are fed into the network 170 where it is processed, stored, and utilized to ensure that surgical assets are always within reach and prepared for immediate use as the need arises in the hospital.

The reliability of the global asset tracking provided in the system 100 is accentuated by the fact that the global trackers 120 are designed to function synchronously with the local trackers 110. This dual-tiered tracking system facilitates a holistic approach to asset management, allowing hospital staff to track the location of carts 160 during transit as well as verify the condition and location of sterilization trays 150 and their contents within more localized settings, such as sterilization facilities and operating rooms.

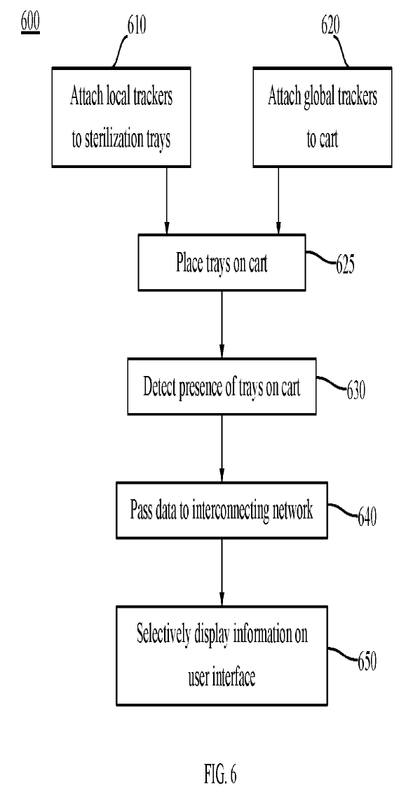

Referring now to FIG. 6, the depicted flowchart provides a portrayal of the operational steps 600 entailed in the asset tracking method within the hospital environment, leveraging both local and global trackers, and coordinated through an interconnected network system. This flowchart delineates the sequential actions that collectively form the process by which surgical tools and trays are tracked, verified, and managed from sterilization to deployment in various hospital procedures.

The first step 610 involves attaching local trackers 110 to each sterilization tray 150. The local trackers 110, resiliently designed to withstand the harsh conditions of autoclave sterilization, facilitate real-time monitoring and verification of the sterilization process. Through embedded temperature sensors and humidity sensors, these local trackers help to ensure the sterility of the tray contents without compromising the protective sterile wrap.

Step 620 entails attaching a global tracker 140 onto a cart 160 used to transport the trays 150 throughout the hospital. The global trackers 140 boast a robust transmission capability to send data in the presence of hospital infrastructural barriers to provide location data of the movable carts 160 to the interconnected network system 170.

In step 625 trays 150 are then placed onto the cart 160, and in step 630 the presence of sterilization trays 150 on the carts 160 are detected. This step is conveniently executed at a strategically positioned checkpoint that houses both local receivers 130 and global receivers 140. When a cart 160 outfitted with both trays 150 and a global tracker 140 passes through this checkpoint, the local receiver verifies the condition and presence of trays 150 via the local tracker, while the global tracker confirms the identity of the cart 160 and associates the present trays 150 with the cart 160. In one embodiment, trays 150 and carts 160 within the checkpoint are distinguished from trays 150 and carts 160 outside the checkpoint by the signal strength (RSSI) of the local trackers 110 and global trackers 120 at the local receiver 130 and global receiver 140, respectively, associated with the checkpoint.

At step 640 collected data is passed into the interconnecting network 170. The interconnecting network 170 aggregates, processes, and utilizes the collected data to provide a comprehensive, up-to-date overview regarding the location, condition, and status of the trays 150 and carts 160 within the hospital. The information gathered by the interconnecting network 170 s facilitates logistical decisions and helps to ensure that surgical instruments are prepared, sterile and present when and where needed.

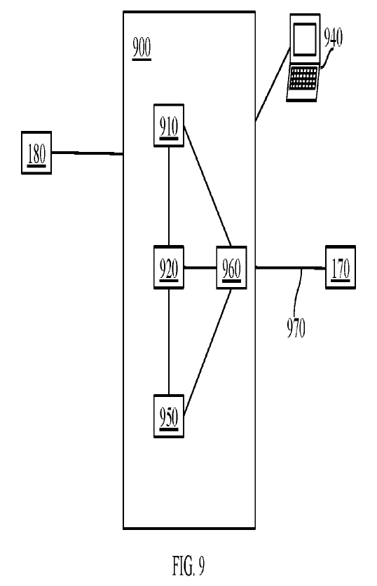

At step 650 the location, condition, status, and other pertinent information regarding the trays 150 and carts 160 are selectively displayed on a user interface 940 (shown in FIG. 9) The user interface 940 enables a user to select trays 150, carts or surgical tools 230 of interest and obtain information relating to its location and sterilization status.

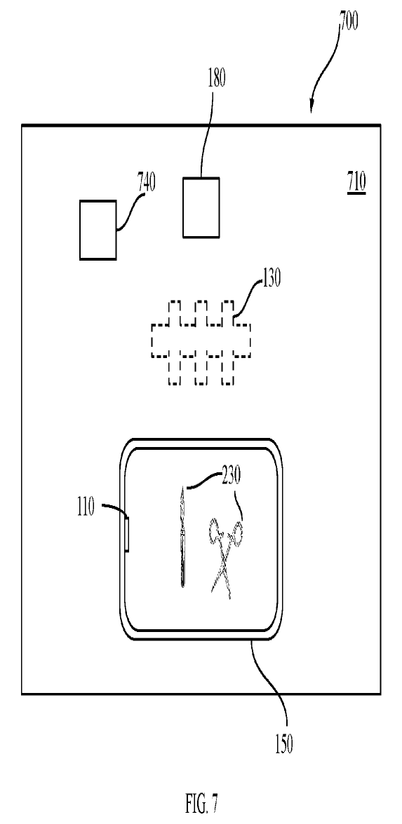

FIG. 7 depicts an assembly table 700, which is advantageously positioned within a sterile processing department or other suitable location within the hospital. The assembly table 700 is pictured from a top view to illustrate its configuration and spatial relationship with other components of the hybrid asset tracking network system 100.

The assembly table 700 comprises a flat horizontal surface 710 upon which sterilization trays 150 can be conveniently placed for loading or unloading of surgical tools. The surface 710 may be formed from a sturdy, non-porous material to support the weight of the trays 150 and to allow for easy disinfection.

Integrated within the assembly table 700 is a local receiver 130, which is inset into the structure of the assembly table 700. The local receiver 130 is specifically adapted to detect the presence and identity of local trackers 110 affixed to sterilization trays 150 that are positioned on the assembly table 700. This integration facilitates uninterrupted communication between the trays 150 and the local receiver 130 despite proximity to metallic surgical tools and the dense arrangement of equipment typically found in assembly areas.

In some embodiments an indicator mechanism 740, which may consist of audible alarms or visual signals such as LED lights, is operatively connected to the local receiver 130. In other embodiments indicator mechanism 740 is operatively connected to optical system 180. This indicator mechanism 740 provides immediate feedback to the hospital personnel when a sterilization tray 150 is correctly positioned on the assembly table 700, thereby improving the process flow and reducing the risk of erroneous tool assembly.

To facilitate the identification process of surgical tools within the trays 150, the local receiver 130 is communicatively coupled to an optical system 180. The real-time data concerning the presence and identification of trays 150 and their content is transmitted to the interconnecting network 170 and subsequently synchronized with the asset database 900 (shown in FIG. 9).

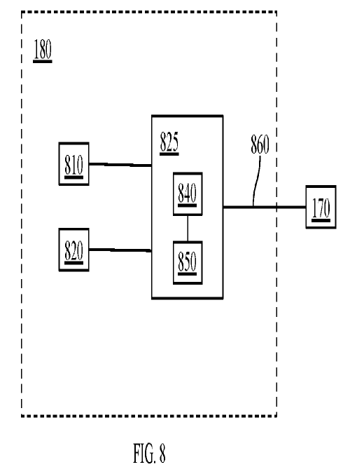

FIG. 8 depicts a schematic representation of an optical system 180 equipped with computer vision capabilities for identifying surgical tools during assembly on the assembly table 700. This system functions as part of the network system 100, providing the ability to verify the presence and composition of sets of surgical tools 230 within each sterilization tray 150 without relying solely on manual inspection.

The optical system 180 comprises one or more high-resolution cameras 810 strategically positioned above the assembly table 700 to capture clear and detailed images of the tools located within the sterilization trays 150. Embedded lighting units 820 may be included to illuminate the tray contents uniformly, thereby enhancing the capability of the cameras 810 to distinguish individual surgical tools 230 based on their physical characteristics such as size, shape, and surface features.

1n recognition of the diverse and intricate nature of surgical tools 230, the optical system's computer vision software is programmed with an extensive library 830 containing tool profiles. This library facilitates the precise identification and categorization of each surgical tool, even under challenging conditions where tools may partially overlap or vary in orientation.

The optical system 180 includes a processing unit 825 that leverages image processing algorithms 840 which process the raw visual data captured by the cameras 810, transforming it into meaningful information regarding tool identification and quantities. These algorithms 840 are capable of discerning even minor visual discrepancies, ensuring that the accuracy of the inventory matches the exact requirements for the corresponding surgical procedures. In one embodiment, optical system further identifies defects in tools resulting from wear or misuse and alerts hospital staff of such defects.

1n an embodiment processing unit 825 dynamically controls lighting units 820 to optimize lighting conditions based on received image data.

Processing unit 825 also includes a machine learning module 850 that facilitates computer vision, which is initially trained on a large corpus of examples and continually refines the recognition process based on newly acquired images and feedback. This module enables the system to adapt to new tool types, variations in tool design, and any changes in the hospital's inventory, reinforcing the system's scalability and flexibility.

A communication interface 860 enables bidirectional interaction between the optical system 180 and the interconnecting network 170. This interface ensures that data recognized and processed by the optical system 180 can be incorporated into the network system 100, thereby maintaining synchronized records of surgical tool inventories.

The alignment between the optical system 180 and the local receiver 130 integrated into the assembly table 700 affords a comprehensive verification mechanism. Upon a sterilization tray 150 being set on the assembly table 700, the local receiver 130 signals the optical system 180 to initiate the identification process. After tools within the tray 150 are identified, the information is then passed through the communication interface 860 to the interconnecting network 170.

The interconnecting network 170, in conjunction with the asset database 900, updates inventory records based on identifications made by the optical system 180. It correlates this information with the specific tray and the status of tools contained therein, thereby enhancing the accuracy of asset tracking and enabling informed decision-making for surgical tool management within the hospital environment.

In practice, the optical system 180 not only streamlines the assembly and verification of surgical tools but also significantly reduces the potential for human error, augments staff efficiency, and contributes to the assurance of adhering to rigorous sterilization protocols. It also provides a reliable, automated recordkeeping system that supports regulatory compliance and audit preparations, ultimately benefiting both the healthcare provider and patient through enhanced operational safety and quality of care.

FIG. 9 depicts a generalized schematic diagram of an asset database 900 interconnected with the optical system 180 for updating and managing inventory records of surgical tools identified during the assembly process.

The diagram illustrates the asset database 900, which includes a centralized database server 910. This server is designed to manage a multitude of records detailing the surgical tools 230. The individual surgical tool 230 records may include identification data, such as the type of tool, quantity, sterilization cycle information, location data within the hospital infrastructure, and usage history. The database server 910 is also configured to store data related to the sterilization trays 150 and carts 160, each equipped with their respective local 110 and global trackers 120.

Asset database 900 also includes a data management system 920, which is responsible for organizing, indexing, and retrieving data efficiently upon request. The database server 910 employs querying functionalities facilitated by the data management system 920 that allow for real-time access to inventory statuses and history, ensuring up-to-date information is available to hospital staff.

The optical system 180 serves as an input source for the asset database 900, providing information regarding the presence and condition of surgical tools 230 within sterilization trays 150. As the optical system 180 identifies and quantifies tools through the use of cameras 810, image processing algorithms 840 and machine learning module 850, this information is conveyed to asset database 900 through the communication interface 860.

Upon receipt of the identification data from the optical system 180, the data management system 920 updates existing inventory records within the database server 910. These updates include modifications to tool quantities, the addition of newly-recognized surgical tools 230, and the flagging of surgical tools 230 for repair or replacement due to identified defects.

Integration with the local receiver 130 associated with the assembly table 700 ensures that the inventory updates are attributed to the correct sterilization tray 150, thereby maintaining integrity within the tracking process. Additionally, this integration enables automatic updates to the inventory records as tools are assembled on or removed from the trays, keeping the tray-specific tool listings current and accurate.

A user interface 940, which may consist of a web portal, a mobile application, or a software module on a hospital network, is provided as a front-end to the asset database 900. This user interface 940 enables authorized hospital personnel to perform searches, view records, generate reports, and receive alerts.

The user interface 940 presents data in a structured and user-friendly manner, allowing hospital staff to quickly ascertain the availability, location, and sterilization status of surgical tools. This facilitates effective planning for surgical procedures, reduces incidences of tool shortages, and enhances the hospital's responsiveness to emergent needs.

Additional features within the asset database 900 include data analytics tools 950, which are utilized to discern patterns in tool usage, track the frequency of sterilization cycles, and predict future inventory needs based on historical data. Such analytics can provide valuable insights for hospital administration, enabling more informed decision-making concerning asset procurement and maintenance schedules.

Security protocols 960 are rigorously applied throughout the asset database 900 to ensure data confidentiality, integrity, and availability. As healthcare facilities are bound by stringent legal standards, such as the Health Insurance Portability and Accountability Act (HIPAA), the database network implements encryption, access controls, and audit trails to protect sensitive information and comply with regulatory frameworks.

Asset database 900 connects with the interconnecting network 170 through network connections 970. The design of asset database 900 emphasizes scalability and interoperability, allowing it to integrate seamlessly with other hospital information systems. This interoperability facilitates synchronization of the inventory records data with electronic health records (EHRs), surgical scheduling systems, and supply chain management platforms, thereby expanding the utility and reach of the asset tracking network within the hospital environment.

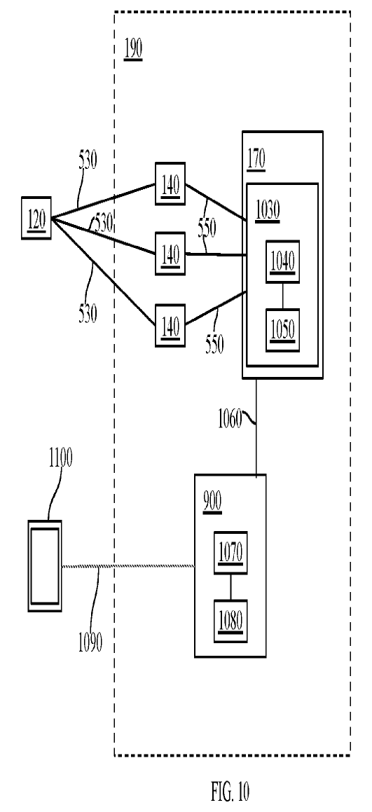

FIG. 10 illustrates the localization system 190 employed in the hybrid asset tracking network system 100, which facilitates the accurate and dynamic location tracking of carts 160, and thus the sterilization trays 150 and surgical tools 230 associated with such carts 160, throughout the hospital premises. The localization system 190 is configured to process and analyze the signal strength of global trackers 120 and update the asset database 900 with real-time location information regarding carts 160.

The localization system 190 is characterized by its ability to receive signals emitted by the global trackers 120 mounted on carts 160. Each global tracker 120 is equipped with a transceiver module 410 that transmits a unique identifier along with signal strength data that is captured by the global receivers 140 spread throughout the hospital. The transceiver module 410 is designed to operate within frequency bands optimal for global tracking, such as UHF or SHF, to ensure efficient propagation of signals over long distances and through various building materials.

Global receivers 140 are equipped with receiver modules 420 which are capable of receiving signals from the global trackers 120 amidst electronic noise and interference commonly present in a hospital environment. Receiver modules 420 have sufficient sensitivity and range to ensure that carts in transit are duly accounted for within the system.

The signal processing module 1030, which resides within the interconnecting network 170 and is an element in the localization process. This module analyzes signal strength (RSSI) from global trackers 120 with respect to one or more global receivers 140 to determine the approximate location of each cart 160. The signal processing module 1030 employs signal processing algorithms to compensate for environmental factors and obstructions that may influence signal propagation.

The position estimation algorithm 1040 within the signal processing module 1030 leverages signal strength data to estimate the positions of carts 160. This algorithm uses techniques such as trilateration or fingerprinting to map signal strength readings to specific locations within the hospital layout. The algorithm's implementation considers the hospital's physical characteristics and the distribution pattern of the global receivers 140 to enhance location precision.

Another feature within the localization system 190 is the synchronization mechanism 1050, which ensures that data from the global trackers 120 is updated in real-time and reflects the latest movements of the carts 160. As carts traverse the hospital, their respective global trackers 120 continuously report signal strength readings that are rapidly processed and converted into updated location status by the synchronization mechanism 1050.

The database interface 1060 represents the connectivity between the signal processing module 1030 and the asset database 900. As the estimated positions of the carts 160 are derived from the signal strength data, the database interface 1060 ensures that this information is promptly reflected in the asset database 900, maintaining a current and accurate representation of cart 160 locations.

Asset database 900 includes location data records 1070, which store the positional information pertaining to each cart 160. The location data records 1070 are cross-referenced with the sterilization status and the inventory of surgical tools 230, providing an integrated overview of assets locations and readiness for use.

Through a facility map integration 1080, the asset database 900 can visually depict the real-time locations of carts 160 against the backdrop of a digital map representing the hospital floor plan. This graphical representation enhances situational awareness and allows hospital personnel to quickly ascertain where assets are located at any given time.

The user interface 940 is in operative communication with the localization system 190 and asset database 900, enabling staff members to perform location searches and receive prompt output regarding the location of specific carts 160, sterilization trays 150 or surgical tools 230. The query system can also be utilized for tracking the flow of assets and identifying potential bottlenecks or inefficiencies in the asset management process.

The localization system 190 may additionally provide mobile device integration 1090 that extends its functionality to hospital portable devices 1100 such as smartphones or tablets. Mobile device integration 1090 allows staff to conveniently access asset location data, providing flexibility and mobility to react to the dynamic needs within the hospital environment. Mobile device integration 1090 may also facilitate the tracking and localization of hospital portable devices 1100, utilizing, for example, the signal strength of a Bluetooth signal emitted by such portable devices 1100.

The localization system 190 embedded within the hybrid asset tracking network system 100 helps to ensure that the hospital staff has continuous, real-time access to accurate location data for carts 160, sterilization trays 150 and surgical tools 230. This capability is useful for optimizing the logistics of surgical tool management and contributes to enhancing the quality of patient care through improved operational efficiency and asset readiness.

Although exemplary embodiments of the present disclosure have been described in detail, those skilled in the art will appreciate that various changes, substitutions and improvements disclosed herein may be made without departing from the spirit and scope of the disclosure in its broadest form.

The description and drawings in the present disclosure should not be read as implying that any particular element, step, function or advantage is an essential element that must be included in the claim scope. The scope of patented subject matter is defined only by the allowed claims.

What is claimed is:

1. An asset tracking system for surgical tools within a hospital environment, comprising:
a plurality of sterilization trays, each tray equipped with a local tracker capable of withstanding temperatures of at least 250° F;
local receivers in communication with local trackers, wherein the local receivers are positioned in proximity to areas where said sterilization trays are sterilized;
carts for transporting said sterilization trays, each cart equipped with a global tracker capable of transmitting data at least 40 meters;
global receivers in communication with global trackers, wherein the global receivers are positioned in a plurality of operating rooms in the hospital environment;
a network in communication with both local receivers and global receivers, wherein the network registers the presence of the sterilization trays on the carts at a checkpoint having both a local receiver and a global receiver.

2. The asset tracking system of claim 1, wherein the local trackers operate on a low frequency band and the global trackers operate on at least one of an ultra high frequency band and a super high frequency band.

3. The asset tracking system of claim 1, further comprising an assembly table with an integrated local receiver for identifying sterilization trays on said assembly table.

4. The asset tracking system of claim 3, further including an optical system with computer vision capabilities for identifying surgical tools within said sterilization trays during assembly.

5. The asset tracking system of claim 4, wherein the system further comprises a database configured to update records of tools in each tray based on identifications made by said optical system.

6. The asset tracking system of claim 1, wherein said local trackers further comprise a temperature sensor to confirm passage of sterilization trays through an autoclave cycle and to verify attainment of a specified sterilization temperature.

7. The asset tracking system of claim 1, wherein the sterilization tray is enclosed by a sterile wrap, and wherein the local trackers further comprise a humidity sensor capable of detecting moisture levels indicative of a wet load condition without disturbing the sterile wrap.

8. An asset tracking method within a hospital environment, comprising the steps of:
attaching local trackers to sterilization trays, said local trackers capable of operating at autoclave sterilization temperatures;
placing global trackers on carts used for transporting said trays;
detecting the presence of said sterilization trays on said carts by registering signals from both local and global trackers at a checkpoint; and
employing a network to collect data regarding the location and status of said trays and carts.

9. The method of claim 8, further including the step of identifying tools within sterilization trays using an optical system with computer vision technology during assembly on an assembly table containing a local receiver.

10. The method of claim 8, wherein the local trackers communicate with the local receivers with a low frequency signal, and the global trackers communicate with global receivers with an ultra high frequency signal.

11. The tracking method of claim 8 further comprising the step of detecting a wet load condition, wherein the detection of a wet load condition is accomplished via humidity sensors integrated into the local trackers.

12. An optically enabled system for verifying contents within sterilization trays, comprising:
an optical system with computer vision capabilities for identifying surgical tools during tray assembly;
a local receiver integrated into an assembly table to detect the presence of a sterilization tray; and
a database networked with said optical system to update and manage inventory records corresponding to identified surgical tools.

13. The system of claim 12, wherein the optical system is programmed to recognize specific surgical tools and update the database with tool types and quantities present in the sterilization tray.

14. A monitoring system for ensuring proper sterilization of surgical tools, the system utilizing: local trackers with integrated temperature sensors to verify sterilization temperatures reached
during autoclave cycles;
local receivers positioned at strategic points to detect signals emitted by said local trackers; global trackers on carts for conveying sterilization trays throughout the hospital vicinity; and
an interconnected network that synchronizes and records data obtained from said temperature sensors, local, and global receivers.

15. The monitoring system of claim 14, wherein said network manages data, including the status, location, and confirmation of sterilization of said surgical tools across different hospital departments and areas.

16. A hospital-wide asset tracking network system for surgical tools, the system comprising: intercommunicating local and global devices affixed to sterilization trays and carts respectively; a plurality of local and global receivers positioned throughout the hospital for capturing signal
strengths to approximate locations;
database infrastructure unified with said receivers for maintaining dynamic records of tray and cart locations; and
processing means to analyze said signal strengths and update said database infrastructure with current location information of trays and carts.

17. The network system of claim 16, wherein said database infrastructure reconciles the actual location with sterilization status to ascertain the readiness of surgical tools for operative procedures.

18. A surgical tray tracking apparatus, comprising:
a local tracker with a ruggedized design to endure local sterilization processes, said tracker affixed to sterilization trays; and
a temperature and humidity sensor integrated within said local tracker to monitor conditions indicating tray sterilization status and potential wet loads.

19. A method for managing sterilization and location data of surgical trays in a hospital, the method employing local tracking with ancillary temperature and humidity sensing coupled with global based cart localization, to provide a user with asset location and sterilization status.

20. The method of claim 19 wherein the local tracking utilizes low frequency signals, and wherein the global based cart localization utilizes ultra high frequency signals.
